(19) United States
(12) Patent Application Publication
(10) Pub. No.: US 2015/0000529 A1
(43) Pub. Date: Jan. 1, 2015

(54) CENTRAL AIR FRESHENER

(71) Applicant: Birdeal Ferguson, North Las Vegas, NV (US)

(72) Inventor: Birdeal Ferguson, North Las Vegas, NV (US)

(21) Appl. No.: 13/932,185

(22) Filed: Jul. 1, 2013

(51) Int. Cl. A61L 9/12; B01D 46/00

(52) U.S. Cl. CPC A61L 9/12; B01D 46/0038
USPC 96/222

(57) ABSTRACT

A central air freshener comprising an air filter, where the air filter includes a filter material to trap a plurality of air pollutants pumped from a central AC unit; a plurality of slats within the air filter, where the plurality of slats support the filter material; and an air freshener attached between at least two of the plurality of slats, where the air freshener provides a fragrance that is distributed as the central AC unit pumps air through a building. The air fresheners are exchangeable once used and to change the fragrance distributed.

### BACKGROUND OF THE INVENTION

1. Field of the Invention

The present invention relates to an air freshener that attaches to the central air filter in a building to add a scent to the air that is circulated.

2. Description of Related Art

Air fresheners are common in households to ensure that the ambient air smells nice and fresh. The air fresheners are available in a variety of different embodiments ranging from infusers, sprays, candles, incense and oils. Typically, the air fresheners are distributed into the various rooms throughout the house to spread the fragrance. In especially large rooms multiple air fresheners are required to evenly distribute the smells. Some air fresheners independently disperse the fragrance like infusers and oils, while others require the user to actively disperse the fragrance like sprays and candles. While the air fresheners are pleasant and effective, it becomes expensive and tedious to continuously replace the used air fresheners.

It would be beneficial in the art to provide an air freshening method that more widely distributes a fragrance throughout the house. It would also desirable in the art to provide an air freshener that is inexpensive and easy to maintain.

### SUMMARY OF THE INVENTION

In view of the foregoing disadvantages inherent in the prior art, the general purpose of the present invention is to provide an air freshener for a central AC unit, configured to include all of the advantages of the prior art, and to overcome the drawbacks inherent therein.

Accordingly, an object of the present invention is to provide an air freshener that attaches to an AC filter in an AC unit to evenly distribute fragrance throughout a home or building.

To achieve the above objects, in an aspect of the present invention, a central air freshener is described comprising an air filter, where the air filter includes a filter material to trap a plurality of air pollutants pumped from a central AC unit; a plurality of slats within the air filter, where the plurality of slats support the filter material; and an air freshener attached between at least two of the plurality of slats, where the air freshener provides a fragrance that is distributed as the central AC unit pumps air through a building. The air fresheners are exchangeable once used and to change the fragrance distributed.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawing in which:

FIG. 1 depicts a perspective view of a central air freshener in accordance with an exemplary embodiment of the present invention.

### DETAILED DESCRIPTION OF THE DRAWINGS

The present invention relates to an air freshener that attaches to the central air filter in a building to add a scent to the air that is circulated. The present invention provides a central air freshener to evenly distribute a fragrance through multiple rooms in a home or building utilizing the central air conditioning system as the distribution source. The central air freshener is composed of two main parts an air filter and an air freshener. The air filter is fitted into the AC vent with the air freshener detachably fastened between the slats. The air freshener may stay in place for up to 30 days allowing for easy and convenient exchange during the use of the filter.

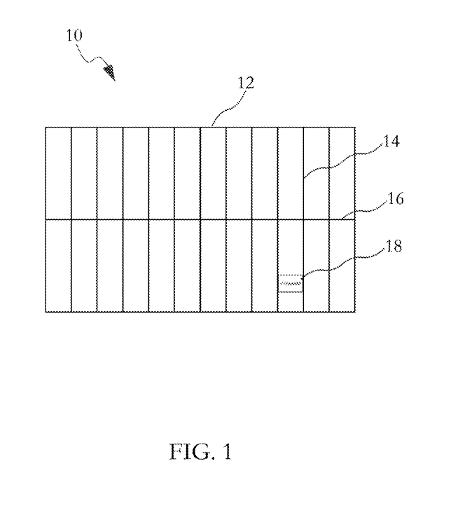

Turning now descriptively to the drawings, referring to FIG. 1, a perspective view of a central air freshener 10 is shown in accordance with an exemplary embodiment of the present invention. The central air freshener 10 includes an air conditioning (commonly referred to as AC) filter 12 and an air freshener 18. The AC filter 12 fits into a central AC unit to catch microscopic smoke particles, pollutants, pet dander, dust, mold spores, and pollen in the pumped air before the air is circulated throughout the house or building. The AC filter 12 typically comprises a square or rectangular shape depending on the specifications of the central air unit. Traversing the AC filter 12 is a plurality of slats 14. The slats 14 provide the structural integrity for the filter material within the AC filter 12. The slats 14 may be made from wire or plastic, and arranged evenly in a horizontal, vertical or even lattice pattern. The AC filter 12 may also include a support wire 16 as additional structural support to the filter 12.

Attached between two of the slats 14 is an air freshener 18. The air freshener 18 clips onto the slats 14, and is removable by unlatching the clips. The air freshener 18 provides a fragrance of the user's choosing which may range from fruits, musk, flowers, spices or a combination thereof. The air freshener 18 may comprise a gel or oil based fragrance for long lasting and permeating smells. Common AC filters 12 are replaced every three to six months. The air freshener 18 may last up to a month. Thus, the removability of the air freshener 18 is beneficial to allow for exchange of the used air freshener 18 with a new one. Also, the user may exchange the fragrances used to match the season, holiday or general preferences.

With the central air freshener 10 a user is provided a unique and easy to use product that works by clipping an air freshener 18 between the slats 14 of an air filter 12. The air filter 12 is then placed within the AC unit to simultaneously trap the pollutant particles pumped and distribute the fragrance throughout the building. One or more air fresheners 18 may be included with each AC filter 12, to replace one air freshener 18 per month that the AC filter 12 is used. For example, if the AC filter 12 lasts for three months then three air fresheners 18 are included with the filter 12. Likewise, if the AC filter 12 lasts for six months then six air fresheners 18 are included. Different fragrances of air freshener 18 may be available with each AC filter 12. The central air freshener 10 is ideal for homes, buildings, hotels and the like which wish to distribute a pleasant scent throughout the structure.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A central air freshener comprising:
a. an air filter, where the air filter includes a filter material to trap a plurality of air pollutants pumped from a central AC unit;
b. a plurality of slats within the air filter, where the plurality of slats support the filter material; and
c. an air freshener attached between at least two of the plurality of slats, where the air freshener provides a fragrance that is distributed as the central AC unit pumps air through a building.

2. The central air freshener according to claim 1, where the air freshener is interchangeable with another air freshener.

3. The central air freshener according to claim 1, where the air freshener is attached with clips.

4. The central air freshener according to claim 1, where the air filter lasts for three months.

5. The central air freshener according to claim 4, where three air fresheners are included with the air filter for exchange every month.

6. The central air freshener according to claim 1, where the air filter lasts for six months.

7. The central air freshener according to claim 6, where six air fresheners are included with the air filter for exchange every month.
